(19) United States
(12) Patent Application Publication
(10) Pub. No.: US 2010/0185179 A1
(43) Pub. Date: Jul. 22, 2010

(54) NEEDLED CANNULA WITH FILTER DEVICE

(75) Inventor: Gregory W. Chan, San Francisco, CA (US)

Correspondence Address: ABBOTT CARDIOVASCULAR SYSTEMS INC./BSTZ;BLAKELY SOKOLOFF TAYLOR & ZAFMAN LLP, 1279 OAKMEAD PARKWAY, SUNNYVALE, CA, 94085-4040, US

(73) Assignee: ABBOTT CARDIOVASCULAR SYSTEMS INC., Santa Clara, CA (US)

(21) Appl. No.: 12/356,835

(22) Filed: Jan. 21, 2009

(51) Int. Cl. A61M 25/06; A61M 1/00

(52) U.S. Cl. 604/508; 604/272; 606/200

(57) ABSTRACT

An apparatus may include a cannula. The cannula may be operable to be percutaneously advanced through a blood vessel. The cannula may have a proximal end and a distal end. At least one needle may be coupled to the distal end of the cannula. A filter device may be coupled at a position proximal to the at least one needle. The filter device may have a distal portion that is operable to have a first diameter under a first condition and a second, different diameter under a second condition. Methods of using such an apparatus are also disclosed.

### BACKGROUND

1. Field

Embodiments of the invention relate to medical devices, or methods of using medical devices. In particular, embodiments of the invention relate to a cannula having a needle and a filter device, or methods of using a cannula having a needle and a filter device.

2. Background Information

In some cases, it is desirable to perform a local treatment at a particular internal site within a patient, as opposed to a systemic treatment. For example, this may be the case when a concentration of one or more substances used to treat the internal site cannot be effectively achieved by introduction of the substance systemically or remotely from the site internal. Moreover, the physician may want to treat the site, such as, for example, a diseased portion of an organ or tissue, without treating healthy portions of the organ or tissue.

The local treatment may involve a physician, surgeon, or other practitioner, delivering one or more substances, such as one or more pharmaceuticals or other treatment agents, to the internal site. In order to achieve such local treatment of the internal site, the practitioner may use one or more catheters, cannula, or other medical devices to be inserted into a patient, in order to navigate to the site and deliver the one or more substances to the site.

### BRIEF DESCRIPTION OF THE SEVERAL VIEWS OF THE DRAWINGS

The invention may best be understood by referring to the following description and accompanying drawings that are used to illustrate embodiments of the invention. In the drawings:

FIG. 1 schematically illustrates an embodiment of a catheter system that includes a cannula, at least one needle, and a filter device.

FIG. 2A is a side view of a cannula and an embodiment of a filter device including a helical spring shown with a contracted diameter. In this embodiment the helical spring is capable of being compressed to expand the diameter of the helical spring.

FIG. 2B is a side view of the cannula and the filter device of FIG. 2A, in which the filter device has an expanded diameter as a result of axial compression of the helical spring.

FIG. 3A is a side view of a cannula and another embodiment of a filter device including a helical spring shown with a contracted diameter. In this embodiment the helical spring is capable of being twisted to expand the diameter of the helical spring and the filter device.

FIG. 3B is a side view of the cannula and the filter device of FIG. 3A, in which the filter device has an expanded diameter as a result of twisting the helical spring.

FIG. 4 schematically illustrates an embodiment of the placement of a distal portion of a catheter system in a heart during an injection of one or more therapeutic agents into a left ventricle.

FIG. 5 schematically illustrates an embodiment of a catheter system that includes at least one needle and at least one aspiration port proximate the at least one needle.

FIG. 6 is a front perspective view of a distal end portion of a cannula illustrating a possible arrangement of a plurality of aspiration ports distributed concentrically around a needle that is extendable/retractable from the distal end of the cannula.

FIG. 7 is a side view illustrating an embodiment of a distal end portion of a cannula having at least one needle and a plurality of aspiration ports distributed along a distal length of the cannula.

FIG. 8 is a cross-sectional view taken along a central axis of an embodiment of a distal end portion of a cannula having at least one needle coupled thereto and at least one aspiration and flush (A/F) port.

FIG. 9 is a side view of a distal end portion of a cannula including an embodiment of a filter device, in which there is at least one aspiration port through an exterior surface of the filter device.

FIG. 10 is a side view of an embodiment of a distal end portion of a cannula having a lumen, at least one aspiration port, and at least one sensor to sense a condition of an aspirated fluid in the lumen.

FIG. 11 schematically illustrates an embodiment of a catheter system that includes a first cannula having at least one needle and a second cannula having a filter device, in which the first cannula is inserted through the second cannula.

FIG. 12 is a flow diagram of an embodiment of a process of using a filter device to restrain and optionally aspirate particles.

### DETAILED DESCRIPTION

In the following description, numerous specific details are set forth. However, it is understood that embodiments of the invention may be practiced without these specific details. In other instances, well-known structures and techniques have not been shown in detail in order not to obscure the understanding of this description.

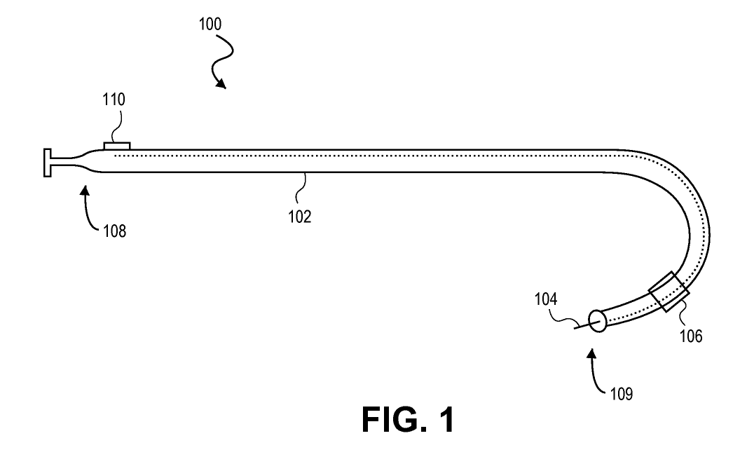

FIG. 1 schematically illustrates an embodiment of a catheter system 100. The catheter system includes a cannula 102, at least one needle 104, and a filter device 106.

The cannula 102 has a proximal end 108 and a distal end 109. The cannula may be sized for and capable of being percutaneously advanced through a blood vessel.

The catheter system also includes the at least one needle 104. The at least one needle is coupled to the distal end of the cannula. The at least one needle may be used to inject one or more therapeutic or treatment agents into a tissue. In some cases, the catheter system may include a plurality of needles coupled to the distal end thereof, such as, for example, to inject different therapeutic agents. The catheter system includes a needle slider 110 to slide the at least one needle.

In some applications, a distal portion of the catheter system having the at least one needle may be percutaneously advanced through a blood vessel and used to inject a therapeutic agent into a tissue, such as, for example, a tissue of the heart. One challenge with such injections is the potential for stroke or embolism under certain conditions. For some injectable therapeutic agents, the potential for stroke or embolism may be especially great in the event of non-engagement or disengagement of the one or more needles. At least for certain therapeutic agents, if the therapeutic agent leaks out of the tissue or site of injection, or is delivered outside of the tissue or site of intended injection, such as by an insufficiently engaged needle, there may be a risk of stroke or embolic hazard. Certain such therapeutic agents may cause thrombus or blood clots that may pose an embolic risk. Other therapeutic agents may gel, coagulate, or otherwise aggregate and thereby pose an embolic risk.

Referring again to the illustration, the catheter system also includes the filter device 106. The filter device is coupled at a position proximal to the at least one needle. The filter device has a distal portion that is operable to have a first diameter under a first condition and a second, different, larger diameter, under a second condition.

Advantageously, including the filter device may help to restrain thrombus, blood clots, injectate, gelled or otherwise aggregated injectate, or other such matter larger than blood cells. Filtering or restraining this matter before it reaches the brain or other peripheral tissues may help to avoid, or at least reduce, the risk of stroke or embolism.

Various filter device designs are contemplated.

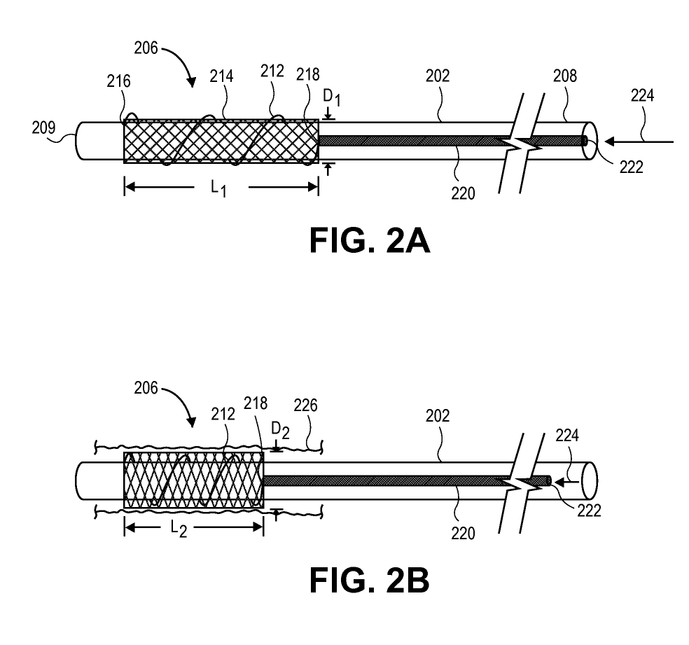

FIG. 2A is a side view of a cannula 202 and an embodiment of a filter device 206 including a helical spring 212 shown with a contracted diameter (D1), in this embodiment the helical spring is capable of being compressed to expand the diameter of the helical spring.

The cannula has a distal end or portion 209 and a proximal end or portion 208. The filter device is coupled to an exterior surface of the cannula at the distal portion of the cannula or toward the distal end. Typically, the filter device is coupled within about 25 cm, often within about 15 cm, of the distal end of the cannula, although this is not required.

The filter device has a frame portion including the helical spring 212. By way of example, the helical spring may include a forged material, a molded material, or a wound material. Often, the helical spring may include or be made of a metal. For example, various metals used in stents are suitable. The term metal is intended to include both pure metals and alloys. Alternatively, the helical spring may be made of a plastic, such as of the types used in stents or like medical devices. If desired, the helical spring may be made of, or include, a shape memory metal or material. A representative helical spring is shown, although other helical springs, such as having different numbers of coils, may be used instead. Notice that in the starting condition the helical spring and the filter device have a first diameter (D1) and a first length (L1) along the axis of the cannula.

A filtration material 214 is coupled to the helical spring or frame portion of the filter device. A portion of the filtration material may be coupled axially around the exterior surface of the cannula. Examples of suitable couplings include, but are not limited to, laser bonding, adhesive bonding, thermal bonding, and mechanical restriction (for example weaving, tying, or sewing the filtration material to the frame portion).

The filtration material has, or is pierced with, a plurality of openings. The openings may allow blood to flow through them, while restraining larger particles or materials. By way of example, the filter material may include, or be made of, natural or synthetic materials, plastics, polymer mesh material, polytetrafluoroethylene, stainless steel, PEBAX 91 (a biocompatible polymer, such as a polyether block amide resin, sold under the trademark PEBAX™ of ATOCHEM CORPORATION, PUTEAUX, FRANCE). Some such materials may be spun or weaved together and fixed to the frame by knots, ties, adhesives, or the like. Another suitable material is a rubber or other material used for a balloon that has holes formed therein. These are just a few examples. Other embolic protection material and filtration materials are also suitable.

The helical spring has a distal end or portion 216 and a proximal end or portion 218. The distal end or portion of the helical spring is coupled to the exterior surface of the cannula at a fixed position. The proximal end or portion of the helical spring is coupled to a distal end or portion of a shaft 220. The couplings may be achieved by laser bonding, adhesive bonding, heat bonding, welding, mechanical connections, or other bonding or coupling techniques.

The shaft may be an elongated, slender, generally cylindrical mandrel, tendon, wire, bar, rod, or like member. By way of example, the shaft may be made of metal or rigid plastic. The shaft has a proximal end or portion 222 that is at or near the proximal end or portion of the cannula. It is not required that the shaft go all the way to the end of the cannula, as shown in the illustration, as long as the proximal end of the shaft is sufficiently accessible to a practitioner to allow the practitioner to push or otherwise actuate the shaft via its proximal end or portion.

A practitioner may press on the shaft in the distal direction shown by arrow 224. The shaft may represent or serve as a plunger that is operable to slide along the axis of the cannula in the distal direction. This may cause the shaft to exert a force on and compress the helical spring to enlarge the diameter of the helical spring and therefore the filter device.

FIG. 2B is a side view of the cannula 202 and the filter device 206 of FIG. 2A, in which the filter device has an expanded diameter (D2) as a result of axial compression of the helical spring 212 as a result of movement of the shaft 220 in the distal direction 224 by a distance (d). In this condition the filter device has a second, greater diameter (D2) and a second, shorter length (L2). The greater diameter (D2) may approximate an inner diameter of a blood vessel 226 at a region of interest. The different diameters of the filter device are a result of different conditions that include different amounts of compression of the helical spring. Notice also that the filter device has a generally tubular shape in the expanded diameter condition.

In embodiments, the expanded diameter of the helical spring and the filter device may be approximately equivalent to an inner diameter of a blood vessel at a region of interest proximal to an injection site. Advantageously, the helical spring may be compressed by different amounts to achieve these different diameters. If desired, latches or locks may be provided to hold the shaft in different positions to keep the diameter of the filter device from changing unintentionally. In this way, substantially all of the blood flowing through the blood vessel may pass through the filter. The filter device may not be a hollow cylinder, but rather may have filter material that is transverse to the direction of blood flow and that substantially spans the entire cross sectional area available for blood flow. By way of example, the filter material may be coupled axially around the cannula, such as, for example, at one or both of the ends of the filter device. The filter device may be used restrain thrombus or other unwanted particles or material having a size greater than an average size of blood cells. For example, a typical red blood cell has a size of approximately 7 micrometers in diameter, and a typical white blood cell has a size of between approximately 7 and 15 micrometers in diameter.

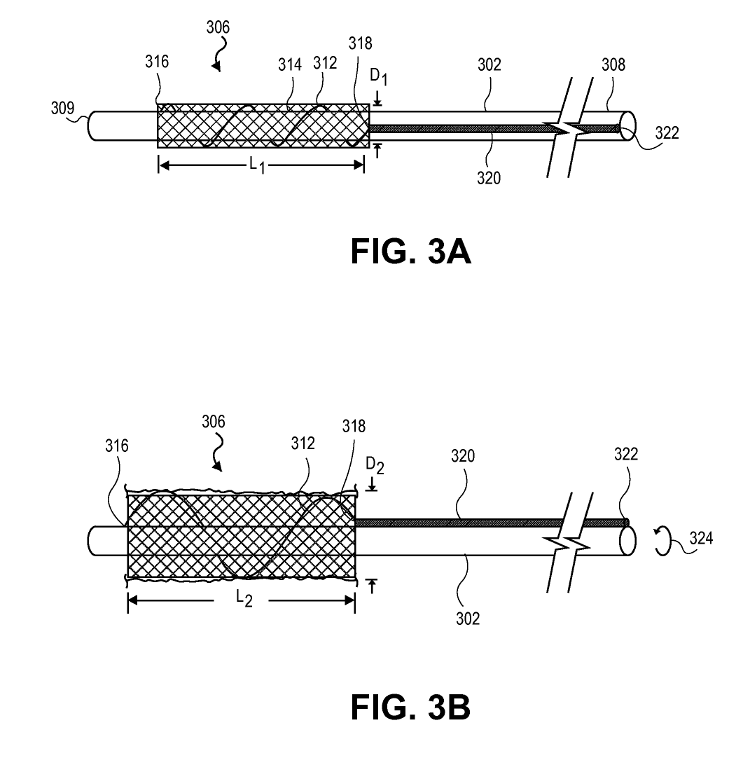

FIG. 3A is a side view of a cannula 302 and another embodiment of a filter device 306 including a helical spring 312 shown with a contracted diameter (D1), in this embodiment the helical spring is capable of being twisted to expand the diameter of the helical spring and the filter device.

This embodiment has features similar to those of the embodiment shown and described for FIGS. 2A-2B. For brevity, all of the similar features will not be repeated. Rather, the discussion will tend to emphasize the new or different features shown in FIGS. 3A-3B.

As before, the filter device is coupled to an exterior surface of the cannula at a distal portion of the cannula. The filter device has a frame portion including the helical spring 312 and a filtration material 314 having a plurality of openings coupled to the helical spring. A distal end or 316 of the helical spring is coupled to the exterior surface of the cannula at a fixed position. A proximal end or portion 318 of the helical spring is coupled to a distal end or portion of a shaft 320. The shaft has a proximal end or portion 322 that is at or near the proximal end or portion 308 of the cannula. The helical spring and the filter device have a first diameter (D1) and a first length (L1) along the axis of the cannula.

Now, in this embodiment, the helical spring may include a torsion spring. The shaft may be operable to rotate or otherwise turn about the cannula to twist the helical spring. A practitioner may turn the shaft about the cannula as shown by arrow 324. This may cause the shaft to exert a force on the proximal end of the helical spring that is attached to the shaft. This may may result in twisting of the helical spring, which may enlarge the diameter of the helical spring and therefore the filter device.

If the rotation or twisting is in the same direction as the coils are wound (tightening of the spring), then the rotation or twisting of the helical spring may cause a decrease in the diameter of the helical spring and filter device. If the rotation or twisting is in the opposite direction as the coils are wound (loosening of the spring), then the rotation or twisting of the helical spring may cause an increase in the diameter of the helical spring and filter device. Advantageously, the helical spring may be twisted by different amounts to achieve different diameters.

FIG. 3B is a side view of the cannula 302 and the filter device 306 of FIG. 3A, in which the filter device has an expanded diameter (D2) as a result of twisting the helical spring 312 as a result of turning the shaft 320 about the cannula. In this condition the filter device has a second, greater diameter (D2) and a second, shorter length (L2). The different diameters are a result of different conditions that include different amounts of twisting of the helical spring. Notice also that the filter device has a generally tubular shape in the expanded diameter condition.

Other filter device designs are also suitable. For example, other suitable filter device designs are disclosed in U.S. Patent Application Publication 2005/0015048 (hereinafter “the '048 publication”), entirely incorporated herein by reference. In brief, FIGS. 7-18 of this publication show cannula having filter devices. FIG. 19 of this publication shows a process for using a filter device to restrain and aspirate particles.

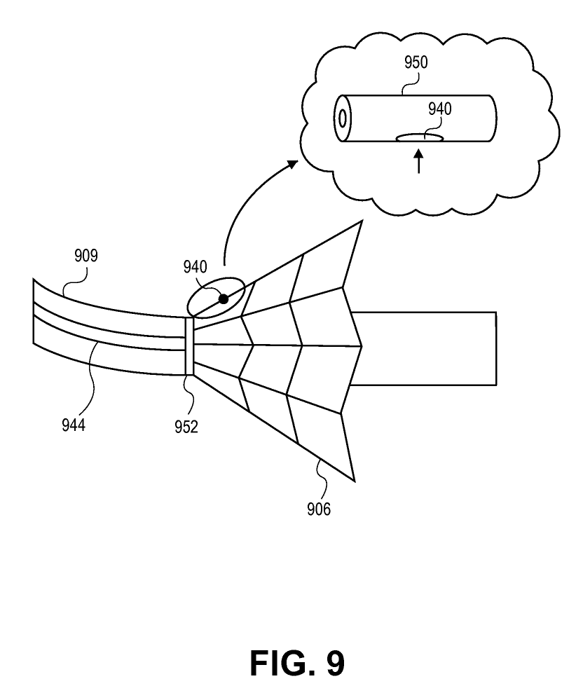

As one example, FIGS. 7-10 of the '048 publication show a filter device and a sheath. The filter device includes a frame portion and a filter material having or pierced by openings that is coupled to the frame portion. The filter device has a proximal end or portion axially coupled to a cannula. FIG. 7 shows the filter device within a sheath. The sheath restrains a distal diameter of the filter device. FIG. 9 shows that the sheath may be retracted in a proximal direction to allow the distal diameter of the filter device to expand. In one aspect, the expanded distal diameter may approximate an inner diameter of a blood vessel or coronary sinus. In various aspects, the filter device may include a shape memory material, self-expanding material, or may expand due to pressure from fluid flow. In this condition, the filter device has a generally conical shape. The sheath may be advanced in a distal direction to at least partially contract the distal diameter of the filter device, such as to allow the cannula having the filter device to be retracted percutaneously.

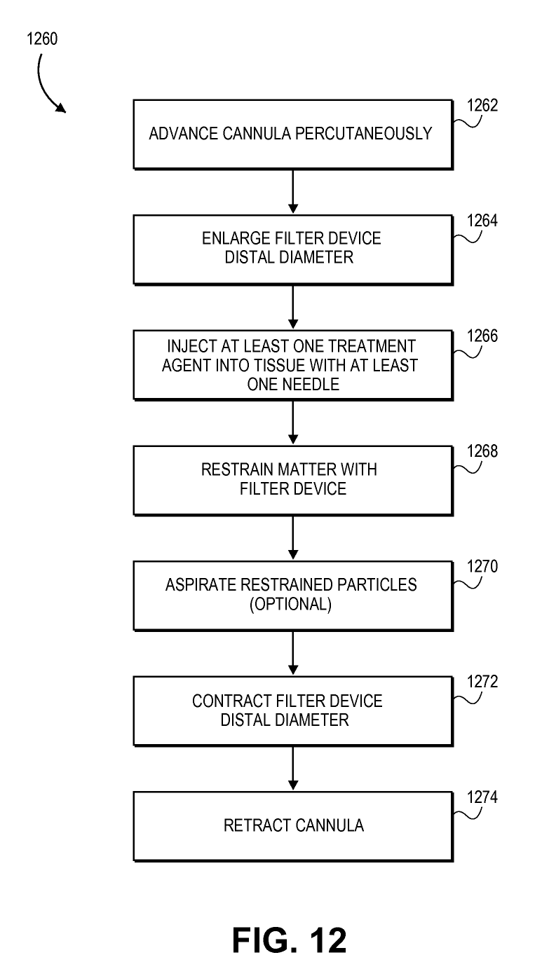

As another example, FIGS. 11-13 of the '048 publication show a cannula, a filter device, and balloons coupled to the filter device and/or the cannula. FIG. 11 shows the filter device with the balloons before they are inflated. In this condition, the distal portion of the filter device has a first, lesser diameter. FIG. 12 shows that the balloons may be inflated to expand the diameter of the distal portion of the filter device. The filter device may optionally include protruding barbs or anchors to engage tissue to help hold the filter device in place, or else may have non-traumatic tips. Again, in this condition, the filter device has a generally conical shape. FIG. 13 shows that the balloons may be deflated to at least partially contract the distal diameter of the filter device. The filter device may potentially include a self-contracting material or shape memory material.

As yet another example, FIGS. 14-16 of the '048 publication show a cannula, a filter device, and tendons that extend through the cannula. The tendons are attached to a distal portion of the filter device. FIG. 14 shows the filter device with a contracted distal diameter. FIG. 15 shows that the tendons may be used to expand the diameter of the distal portion of the filter device. FIG. 16 shows that the tendons may be used to at least partially contract the distal diameter of the filter device.

For brevity, just a few of the details of the previously disclosed filter devices have been repeated herein. Further details, if desired, are available in the '048 publication, which has been incorporated herein by reference. Any of these aforementioned filter devices may potentially be used in the medical devices disclosed herein.

Now, in one or more embodiments, a catheter system, such as that shown in FIG. 1, may be an intra-myocardial needle injection catheter system that is used to inject one or more treatment agents into a tissue of the heart. For example, in an embodiment, a catheter system may be used to inject one or more treatment agents into the left ventricle of the heart.

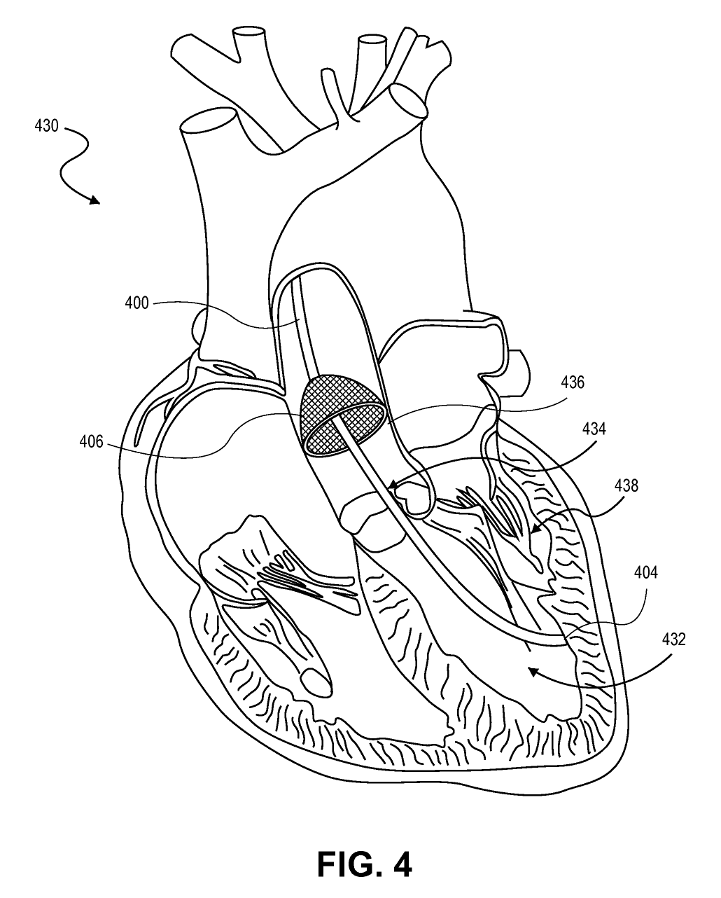

FIG. 4 schematically illustrates an embodiment of the placement of a distal portion of a catheter or cannula system 400 in a heart 430 during an injection of one or more therapeutic agents into a left ventricle 432.

The cannula system 400 may be introduced to the patient vasculature through a femoral artery access point (not shown). The cannula system may be advanced up the aorta, around the aortic arch, passed through the ascending aorta 436, passed through the aortic valve 434, and into the left ventricle 432. Once inside the left ventricle, the cannula may be manipulated by the operator, such as by applying torque, translating the cannula system, and deflecting the distal tip of the cannula system to achieve tip-to-wall contact at the desired location on the left ventricular wall.

At least one needle 404 coupled to a distal end of the cannula is positioned against the wall of the left ventricle. The at least one needle is inserted into the myocardial tissue of the left ventricle to deliver one or more therapeutic agents therein. By way of example, the therapeutic agents may include stem cells, genes, or growth factors, although the scope of the invention is not limited to the delivery of just these types of therapeutic agents.

In the illustration, the filter device 406 is positioned just proximally of the aortic valve 434 in the ascending aorta 436. The illustrated filter device has an expanded diameter that provides full apposition to the aortic wall so that all of the blood passing through the aorta is filtered by the filter device. By way of example, the inner diameter of the aorta for an adult typically ranges from about 1.5 centimeters (cm) to about 2 cm.

However, the scope of the invention is not limited to just this particular position for the filter device. In embodiments of the invention, if the at least one needle were positioned to inject a treatment agent into the left ventricle of a representative patient for which the catheter system was designed to treat, then the filter device would have a position ranging from a carotid artery proximal to the aorta to papillary muscles 438 that are distal to the aortic valve 434. In an embodiment of the invention, the filter device would have a position ranging from the carotid artery to the leaflets of the aortic valve. It is in these positions that the diameter of the filter device would be expanded or enlarged to approximately equal that of the surrounding blood vessel or anatomical features. Typically the filter device should be positioned away from the leaflets of the aortic valve so as not to substantially interfere with their operation. As used herein, designed to treat a representative patient means that the design of the catheter system assumes something about the size of the patient it is intended to treat. For example, the representative patient may refer to a typical or average sized adult.

In other embodiments, the catheter system may be used to inject one or more treatment agents into the right ventricle of the heart. In such embodiments, if the at least one needle were positioned to inject a treatment agent into the right ventricle of a representative patient for which the catheter system was designed to treat, then the filter device may be positioned appropriately to occlude the pulmonary artery outflow tract.

It is not required that a needle injection catheter system include a filter device. In one or more other embodiments, a needle injection catheter system may include at least one hole or other aspiration port to aspirate or remove thrombus, stray injectate, or other unwanted material.

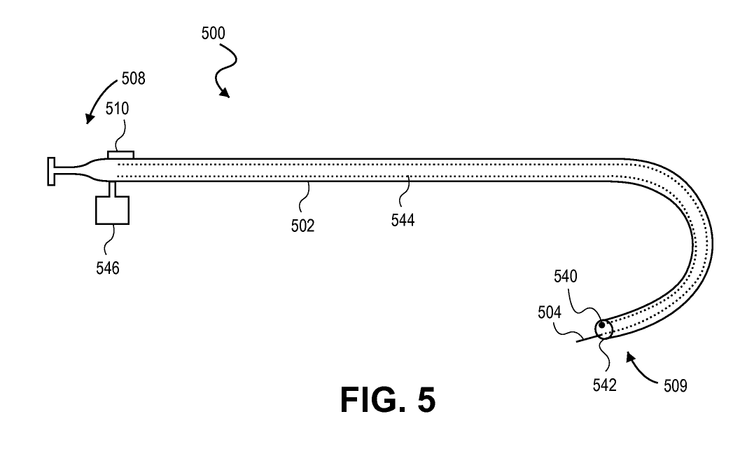

FIG. 5 schematically illustrates an embodiment of a catheter system 500. The catheter system includes at least one needle 504 and at least one hole or other aspiration port 540 proximate the at least one needle.

The catheter system includes a cannula 502. The cannula has an exterior surface, a proximal end 508, and a distal end 509. As previously discussed, the cannula may be percutaneously advanced through a blood vessel.

The catheter system also includes the at least one needle 504 coupled to the distal end 509 of the cannula. The at least one needle may be used to inject one or more therapeutic agents into tissue. In some cases, the catheter system may include a plurality of needles coupled to the distal end thereof, such as, for example, to inject different therapeutic agents. The catheter system includes a needle slider 510 to slide the at least one needle.

The catheter system also includes the at least one hole or other aspiration port 540. The at least one hole or other aspiration port is proximate the at least one needle. As used herein, the at least one aspiration port is proximate the at least one needle if they are within 10 millimeters (mm) and they are very proximate if they are within 3 mm.

As shown, the at least one aspiration port may be on a distal end face 542 of the cannula where the at least one needle is coupled. The distal end face often has a diameter on the order of 10 French or less, and accordingly the aspiration port is generally within 2 mm or less from the needle. Advantageously, placing the at least one aspiration port on the distal end face very proximate the needle may help to remove stray injectate not properly injected into the tissue, injectate that leaks out of the tissue, and/or thrombus resulting from the injection. In one or more embodiments, the at least one aspiration port is within 3 mm or less from the needle. In various embodiments, the aspiration port may have a diameter or other cross-sectional dimension typically ranging from about 20 to 1000 micrometers, often ranging from about 30 to 500 micrometers, sometimes ranging from about 50 to 200 micrometers.

A lumen 544 runs through the cannula from the at least one aspiration port to the proximal portion of the cannula. A vacuum, syringe, or other suction device 546 may be coupled with the proximal portion of the cannula. The suction device may be permanently coupled, or alternatively the cannula may have a connector to allow the suction device to be coupled and decoupled. The lumen may convey the suction provided by the suction device at the proximal portion of the cannula to the at least one aspiration port. The suction may be used to aspirate or remove unwanted materials.

Advantageously, the at least one aspiration port may help to aspirate or remove thrombus, blood clots, injectate, gelled or otherwise aggregated injectate, or other such matter. This may help to reduce the likelihood of stroke or embolism.

Another potential benefit of locating the at least one aspiration port on the distal end face of the cannula where the at least one needle is coupled is the potential that the suction provided through the at least one aspiration port may help to improve the injection of the needle and the therapeutic agent into the tissue. The suction may help to encourage good coupling or contact between the at least one needle and the tissue. Some tissues, such as, for example, myocardial or ventricular tissues, may expand, contract, or otherwise move, such as due to the cardiac cycle. The suction provided by the at least one aspiration port may help to encourage good coupling or contact between the needle and the tissue where the needle is injected. This may help to promote injection of the therapeutic agent at a more consistent and predictable needle engagement depth in the tissue especially when the tissue is moving. This may also help to reduce unwanted perforation of the myocardial wall or other tissue and the possibility of subsequent pericardial effusion or tamponade.

The illustrated catheter system does not have a filter device in order to emphasize that aspiration ports and filter devices may optionally be used separately. However, other embodiments are contemplated in which a needle injection catheter system or cannula includes both a filter device and at least one aspiration port (for example see FIG. 11).

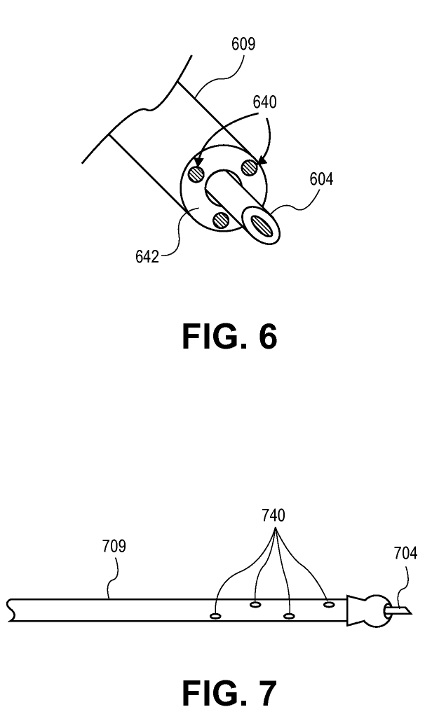

FIG. 6 is a front perspective view of a distal end portion of a cannula 609 illustrating a possible arrangement of a plurality of holes or other aspiration ports 640 distributed concentrically around a needle 604 coupled with the distal end of the cannula.

The plurality of aspiration ports are concentrically distributed around the at least one needle. The plurality of aspiration ports reside at or on a distal end face 642 of the cannula or catheter where the at least one needle is coupled. Advantageously, both of these features may help to improve the aspiration or removal of unwanted material originating from, or as a result of, the needle.

In the illustration, three aspiration ports are shown, although in alternate embodiments, fewer or more aspiration ports may optionally be used. In the illustration, circular holes or aspiration ports are shown, although in alternate embodiments square, oval, rectangular, c-shaped, concentric, or other shaped openings may optionally be used.

FIG. 7 is a side view illustrating an embodiment of a distal end portion of a cannula 709 having at least one needle 704 and a plurality of holes or other aspiration ports 740 distributed along a distal length of the cannula.

As shown, the plurality of aspiration ports may be concentrically distributed around the exterior surface of a distal length of the cannula. The aspiration ports may begin at, adjacent, or at least near, the distal end of the cannula. The aspiration ports may extend a distance in the proximal direction away from the distal end of the cannula. The scope of the invention is not limited to any known distance, although typically the distance is less than 10 cm, often less than 3 cm, sometimes less than 2 cm, sometimes less than 1 cm.

The illustration shows a particular number of ports, although fewer or more ports may optionally be used. Likewise, the illustration shows a particular arrangement of the ports, although other arrangements are also contemplated. If desired, the number of ports, or the number of ports per unit length of the cannula, may optionally increase with increasing proximity to the distal end of the cannula. In the illustration, circular ports are shown, although in alternate embodiments square, oval, rectangular, or other shaped openings may optionally be used.

In some embodiments, an annular or at least partially enclosed space may exist between a portion of a needle and a portion of a distal end of a cannula where the needle is coupled. One potential challenge is that blood may enter this annular or at least partially enclosed space and may tend to clot, which may tend to pose a potential embolic risk.

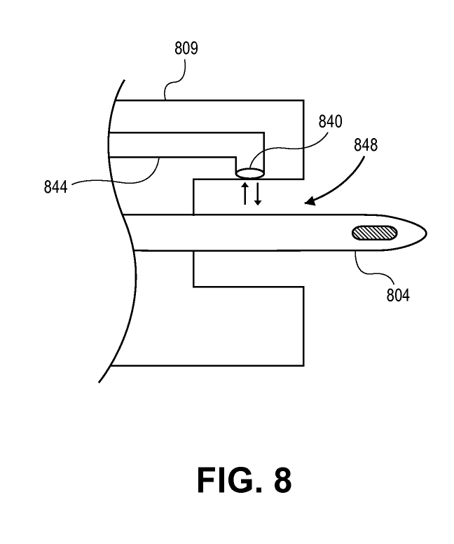

FIG. 8 is a cross-sectional view taken along a central axis of an embodiment of a distal end portion of a cannula 809 having at least one needle 804 coupled thereto and at least one aspiration and flush (A/F) port 840.

The cannula has the at least one A/F port 840. The at least one A/F port is proximate the at least one needle. As used herein, the at least one aspiration port is proximate the at least one needle if they are within 5 mm. The cannula also has at least one lumen 844 running through it from the at least one A/F port to a proximal portion of the cannula (at left in the illustration).

The at least one lumen is configured to alternately aspirate or draw a material inwardly through the at least one hole or other A/F port, and flush or flow a fluid outwardly through the at least one hole or other A/F port. This may be achieved in different ways. In one example embodiment, the at least one lumen may include a single lumen coupled to both a suction device, such as a syringe, to provide suction for aspiration and alternately a pressure increasing device, such as a syringe or pump, to provide pressure to flow a flushing fluid. In another example embodiment, the at least one lumen may include a first lumen coupled to a suction device to provide suction for aspiration and a second lumen coupled to a pressure increasing device to alternately provide pressure to flow a flushing fluid, where both of the lumen are coupled to the at least one A/F port.

As shown, in one or more embodiments, an annular or at least partially enclosed space 848 may exist between a portion of the needle and a portion of the distal end of the cannula. As previously mentioned, one potential challenge is that blood may enter this annular or at least partially enclosed space and may tend to clot. A blood clot formed in this space may tend to inhibit aspiration and/or may dislodge and pose an embolic hazard.

As shown, in one or more embodiments, the A/F port may be located or positioned in this annular or at least partially enclosed space and/or be located or positioned elsewhere but located or positioned to flush or flow a fluid outwardly from the lumen into this annular or partially enclosed space. Advantageously, such flushing of this annular or at least partially enclosed space may help to reduce entry of blood into this annular or at least partially enclosed space and/or clotting of blood in this annular or at least partially enclosed space. This may help to promote good aspiration and may help to lessen the embolic hazard.

In one or more embodiments, the flushing fluid may include a heparinized saline solution, other anticoagulant solution, or other anticlotting solution. Advantageously, use of such an anticoagulant or anticlotting solution may further help to prevent coagulation or clotting of blood in the annular or at least partially enclosed space. Alternatively, simple saline solutions or other suitable flushing solutions may optionally be used.

In an alternate embodiment, a hole or port similar to the A/F port may be used for either aspiration or flush, but not both aspiration and flush.

FIG. 9 is a side view of a distal end portion of a cannula 909 including an embodiment of a filter device 906, in which there is at least one hole or other aspiration port 940 through an exterior surface of the filter device. In some cases there may be a plurality of aspiration ports through the filter device.

Note that the illustrated cannula 909 and the filter device 906 are similar to those of FIG. 9 of the '048 publication. Alternatively, other filter device designs as described herein may be used.

The cannula includes a lumen 944. The lumen runs through the cannula from a proximal portion or end of the cannula to a distal end or portion of the cannula. The lumen is in fluid communication with the at least one aspiration port through the exterior surface of the filter device.

There are different ways of providing fluid communication between the lumen of the cannula and the at least one aspiration port through the filter device. In the illustration, an expanded view of a portion of the filter device having the at least one aspiration port is used to show one approach. As shown, in one or more embodiments, the portion of the filter device having the at least one aspiration port may include a slender metal tube or tubular structure 950, such as, for example, a hypotube. The tubular structure may have at least one hole, representing at least one aspiration port, through an outer wall thereof leading to an inside of the tubular structure. The inside of the tubular structure may serve as an aspiration lumen that is integrated with the filter device. A proximal end of the tubular structure may be in fluidic communication or coupling with the lumen of the cannula. Alternatively, other lumens, conduits, or channels may couple the at least one aspiration port through the filter device with the lumen of the cannula.

It is not required to directly couple the lumen of the cannula to the tubular structure, lumen, channel, or conduit of the filter device. As shown, the filter device may optionally have a manifold 952. The manifold may fluidically couple the lumen of the cannula with tubular structures, lumen, conduits, or channels of the filter device leading to the at least one aspiration port or ports. In one aspect, the manifold may have an inlet coupled with the lumen of the cannula and a plurality of outlets each coupled with a different tubular structure, lumen, conduit, or channel of the filter device to provide suction to a plurality of aspiration ports dispersed at different locations on the filter device.

The lumen of the cannula may convey or provide suction provided at the proximal portion or end of the cannula to the at least one hole or other aspiration port through the filter device. This suction may be used to aspirate or withdraw thrombus, or other unwanted particles or materials through the at least one aspiration port through the filter device. Accordingly, the filter device may incorporate a suction or aspiration mechanism or means. Advantageously, integrating the at least one aspiration port direction with the filter device may help to promote direct aspiration or removal of material restrained by the filter device.

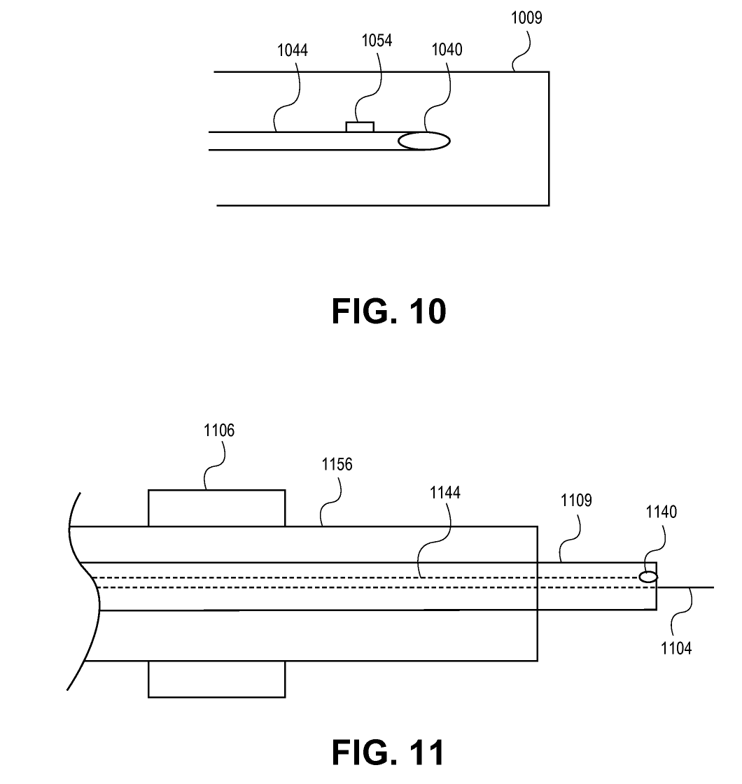

FIG. 10 is a side view of an embodiment of a distal end portion of a cannula 1009 having a lumen 1044, at least one hole or other aspiration port 1040, and at least one sensor 1054 to sense a condition of an aspirated fluid in the lumen.

The cannula, the lumen, and the aspiration port may be substantially as previously shown and described.

The sensor or sensing device may be in the lumen, at least partially in the lumen, or adjacent to the lumen. The sensor may sense a condition of a fluid in the lumen. By way of example, the at least one sensor may sense at least one of a pressure of a fluid in the lumen and a flowrate of the fluid in the lumen.

In one aspect, the sensor may include a MEMS-based pressure sensor. MEMS-based pressure sensors are available from various sources, such as, for example, from Freescale Semiconductor, of Austin, Tex. In another aspect, the sensor may include a piezoelectric pressures sensor. In yet another aspect, the sensor may include a MEMS flow sensor. MEMS flow sensors are known in the art, such as, for example, from U.S. Pat. No. 7,337,678.

The sensor may help to allow confirmation of suction, vacuum, sub-atmospheric pressure, or flow of fluid in the lumen. Advantageously, this may help to allow confirmation that the aspiration is working properly.

FIGS. 1 and 5 show that needled catheter systems may either have filter devices or aspiration ports. However, it is also possible for a needled catheter system to have both a filter device and at least one aspiration port distal to the filter device. For example, the filter device 106 of FIG. 1 may be included in the catheter system 500 of FIG. 5 which has the at least one aspiration port 540. One potential advantage is that the aspiration port may help to aspirate additional material and/or material restrained by the filter device that becomes dislodged, such as, for example, when the filter device is contracted or moved. In addition, the apparatus disclosed herein may optionally incorporate other features, such as, for example, the occlusion devices, or other features as described in the '048 publication. By way of example, the occlusion devices may include balloons, tapered balloons, ePTFE balloons, as described in the '048 publication.

Additionally, instead of a single catheter or cannula, two or more catheters or cannulas may optionally be used together. For example, a guide catheter or cannula and a delivery catheter or cannula may optionally be used together.

FIG. 11 is a cross sectional view of an embodiment of a distal end portion of a catheter system 1109 that includes a distal end portion of a first cannula 1109 having at least one needle 1104 coupled thereto, and a distal end portion of a second cannula 1156 having a filter device 1106 coupled thereto. In the illustration, the first cannula is inserted through the second cannula, although the cannula may be otherwise coupled together.

The at least one needle 1104 is coupled to the distal end portion of the first cannula 1109. In one or more embodiments, the first cannula may include a delivery catheter or cannula. As shown, at least one aspiration port 1140 may be coupled to the first cannula proximate the at least one needle. A lumen 1144 through the first cannula may provide suction to the at least one aspiration port.

The filter device 1106 is coupled at a position proximal to the at least one needle. The filter device is coupled to an exterior surface of the distal end portion of the second catheter or cannula 1156. In one or more embodiments, the second cannula may include a guide catheter or cannula. The filter device has a distal portion that is operable to have a first diameter under a first condition and a second, different diameter under a second condition.

Notice that the filter device and the at least one aspiration port reside on or otherwise correspond to different cannulas or catheters. One potential advantage of coupling the filter device to the second cannula (e.g., a guide catheter or cannula), and coupling the at least one aspiration port to the first cannula (e.g., the delivery catheter or cannula), is that this configuration may allow improved movement or positioning of the needle relative to the intended injection site without having to move the filter device out of place.

Kits including multiple uncoupled or unassembled cannula are also contemplated. One of the cannula may have at least one needle coupled to a distal end thereof, and potentially at least one aspiration port, as described elsewhere herein. Another of the cannula may have a filter device coupled thereto as described elsewhere herein. In one aspect, multiple filters of different sizes, constructions, or actuation mechanism may optionally be provided. In one aspect, a suction device may optionally be included to provide suction to an aspiration port. The kits may optionally include instructions, such as instructions on how to assemble the components of the kits and/or on how to use the assembled components, such as how to use the filter device and/or the aspiration port. The instructions may be written instructions on paper or instructions stored on a compact disk or other machine-readable medium. The kits may be sealed in a manufacturer's sealed package or packaging.

FIG. 12 is a flow diagram of an embodiment of a process 1260 of using a filter device to restrain and optionally aspirate particles.

At block 1262, at least one cannula, such as, for example, cannula 102, may be advanced percutaneously through a blood vessel. The cannula may have a proximal end and a distal end. At least one needle may be coupled to the distal end of the at least one cannula. The filter device may be coupled to the at least one cannula at a position proximal to the at least one needle. It is the distal end that is advanced percutaneously through the blood vessel. In one aspect, the distal end of the cannula may be advanced via a retrograde advancement, such as by being pushed up or down a blood vessel (e.g., such as a blood vein or artery) against or with a flow of blood. Specifically, the cannula may be advanced, such as from one blood vessel into a smaller blood vessel to provide retrograde infusion treatment, to a region of interest such as a region in a coronary sinus, left or right ventricle, or other portion of a heart of a subject.

At block 1264, a distal diameter, or other axial cross-sectional dimension, of a filter device may be enlarged. This is generally performed when the distal end of the cannula is near the desired location. The filter device may have a distal portion that is operable to have, or capable of having, a first diameter under a first condition and a second, different diameter under a second condition. By way of example, in embodiments, the enlargement may include compressing a helical spring of the filter device, or twisting a helical spring of the filter device, although this is not required. The enlarged diameter or dimension may be approximately equivalent to an inner diameter of a blood vessel, or other cross-section available for blood flow, in order to filter substantially all of the blood flowing through this cross-section.

At block 1266, at least one treatment agent may be injected into an internal tissue of a patient with at least one needle. This is commonly performed after the enlargement of the distal diameter, although this is not required. The distal diameter may alternatively be enlarged concurrently with or soon after the injection. In one embodiment, the at least one treatment agent may be injected into a left ventricle, and the distal diameter of the filter device may be enlarged at a position ranging from a carotid artery to papillary muscles that are distal to an aortic valve, or ranging from the carotid artery to leaflets of the aortic valve. However, the scope of the invention is not limited to injection into the left ventricle.

At block 1268, thrombus, gel, aggregated injectate, pieces of tissue, particles, or other material may be restrained with the filter device. In one aspect, the material restrained may have a size greater than an average size of blood cells contained in blood flowing through the filter device.

At block 1270, the restrained particles may optionally be aspirated. However, this is optional, since it is not required that both a filter device and aspiration be used together. If the aspiration is desired, in embodiments, the aspiration may be achieved through at least one aspiration port proximate the at least one needle. In one or more embodiments, the aspiration may include aspirating material concentrically around the at least one needle. In one or more embodiments, the aspiration may include aspirating a material through at least one aspiration port through an exterior surface of the filter device. In one or more embodiments, the aspiration may include alternately flushing a region including the at least one needle and aspirating the region including the at least one needle. In one or more embodiments, in addition to the aspiration, a condition of the aspirated material may be sensed with a sensor. By way of example, the condition may include a pressure of the aspirated material and a flowrate of the aspirated material.

At block 1272, the distal diameter of the filter device may typically be contracted. This means at least partially contracted, since it is not required that the distal diameter be fully contracted.

At block 1274, the cannula and the filter device may be retracted. For example, the cannula may be retracted or withdrawn percutaneously back through the blood vessel through which it was previously advanced and out of the patient.

A particular method has been described to illustrate certain concepts. Operations may optionally be added to and/or removed from the methods. For example, aspiration and/or contraction of the distal diameter may optionally be removed. The operations of the methods may also often optionally be performed in different order. For example, enlargement may be after injection. Many modifications and adaptations may be made to the methods and are contemplated.

In the above description and the claims below, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still co-operate or interact with each other, such as through one or more intervening elements.

In the description above, for the purposes of explanation, numerous specific details have been set forth in order to provide a thorough understanding of the embodiments of the invention. It will be apparent however, to one skilled in the art, that one or more other embodiments may be practiced without some of these specific details. The particular embodiments described are not provided to limit the invention but to illustrate it. The scope of the invention is not to be determined by the specific examples provided above but only by the claims below. In other instances, well-known structures, devices, and operations have been shown in block diagram form or without detail in order to avoid obscuring the understanding of the description.

It will also be appreciated, by one skilled in the art, that modifications may be made to the embodiments disclosed herein, such as, for example, to the sizes, shapes, configurations, forms, functions, materials, and manner of operation, and assembly and use, of the components of the embodiments. All equivalent relationships to those illustrated in the drawings and described in the specification are encompassed within embodiments of the invention.

For simplicity and clarity of illustration, elements illustrated in the figures have not necessarily been drawn to scale. For example, the dimensions of some of the elements are exaggerated relative to other elements for clarity. Further, where considered appropriate, reference numerals or terminal portions of reference numerals have been repeated among the figures to indicate corresponding or analogous elements, which may optionally have similar characteristics.

It should also be appreciated that reference throughout this specification to “one embodiment”, “an embodiment”, or “one or more embodiments”, for example, means that a particular feature may be included in the practice of the invention. Similarly, it should be appreciated that in the description various features are sometimes grouped together in a single embodiment, Figure, or description thereof for the purpose of streamlining the disclosure and aiding in the understanding of various inventive aspects. This method of disclosure, however, is not to be interpreted as reflecting an intention that the invention requires more features than are expressly recited in each claim. Rather, as the following claims reflect, inventive aspects may lie in less than all features of a single disclosed embodiment. Thus, the claims following the Detailed Description are hereby expressly incorporated into this Detailed Description, with each claim standing on its own as a separate embodiment of the invention.

What is claimed is:

1. An apparatus comprising:
a cannula to be percutaneously advanced through a blood vessel, the cannula having a proximal end and a distal end;
at least one needle coupled to the distal end of the cannula; and
a filter device coupled at a position proximal to the at least one needle, the filter device having a distal portion that is operable to have a first diameter under a first condition and a second, different diameter under a second condition.

2. The apparatus of claim 1, wherein the apparatus comprises an intra-myocardial needle injection catheter, and wherein if the at least one needle were positioned to inject a treatment agent into a left ventricle of a heart, then the filter device would have a position ranging from a carotid artery to papillary muscles that are distal to an aortic valve.

3. The apparatus of claim 2, wherein the position of the filter device would range from the carotid artery to leaflets of the aortic valve.

4. The apparatus of claim 1, wherein the filter device comprises a frame portion including a helical spring and a filter material having a plurality of openings coupled to the frame portion, and wherein the filter device has a generally tubular shape under the second condition.

5. The apparatus of claim 4, wherein a distal portion of the frame portion is coupled to the apparatus at a fixed position, and further comprising a shaft having a proximal end and a distal end that is coupled to a proximal portion of the frame portion.

6. The apparatus of claim 5, wherein the shaft comprises a plunger that is operable to compress the helical spring, and wherein the first and second conditions comprise different amounts of compression of the helical spring.

7. The apparatus of claim 5, wherein the helical spring comprises a torsion spring, wherein the shaft is operable to turn about the cannula to twist the helical spring, and wherein the first and second conditions comprise different amounts of twisting of the helical spring.

8. The apparatus of claim 1, further comprising:
at least one aspiration port proximate the at least one needle;
a lumen through the cannula from the at least one aspiration port to a proximal portion of the cannula to convey suction provided at the proximal portion of the cannula to the at least one aspiration port.

9. The apparatus of claim 8, wherein the at least one aspiration port comprises at least one aspiration port on a distal end face of the cannula where the at least one needle is coupled.

10. The apparatus of claim 8, wherein the at least one aspiration port comprises a plurality of aspiration ports distributed around the at least one needle.

11. The apparatus of claim 10, wherein the plurality of aspiration ports are distributed concentrically around the at least one needle at a distal end face of the cannula where the at least one needle is coupled.

12. The apparatus of claim 10, wherein the at least one aspiration port comprises a plurality of aspiration ports distributed along a distal segment of the cannula.

13. The apparatus of claim 8, wherein the at least one aspiration port comprises an aspiration/flush port, and wherein the aspiration/flush port is coupled with at least the lumen to alternatively flow a fluid outwardly through the aspiration/flush port and to draw a material inwardly through the aspiration/flush port.

14. The apparatus of claim 8, further comprising at least one sensor to sense at least one of pressure of a fluid in the lumen and flow rate of the fluid in the lumen.

15. The apparatus of claim 1, further comprising:
at least one aspiration port through an exterior surface of the filter device; and
a lumen from a proximal portion of the apparatus and in fluid communication with the at least one aspiration port to convey suction provided at the proximal portion of the apparatus to the at least one aspiration port.

16. The apparatus of claim 1, wherein the filter device is coupled to an exterior surface of the cannula.

17. The apparatus of claim 1, further comprising a second cannula through which the cannula is inserted, and wherein the filter device is coupled to an exterior surface of the second cannula.

18. A method comprising:
advancing a cannula percutaneously;
enlarging a distal diameter of a filter device;
injecting at least one treatment agent into a tissue with at least one needle;
restraining matter with the filter device;
contracting the distal diameter of the filter device; and
retracting the cannula.

19. The method of claim 18, wherein said injecting comprises injecting the at least one treatment agent into a left ventricle, and wherein said enlarging comprises enlarging the distal diameter of the filter device at a position ranging from a carotid artery to papillary muscles that are distal to an aortic valve.

20. The method of claim 19, wherein the position ranges from the carotid artery to leaflets of the aortic valve.

21. The method of claim 18, wherein said enlarging comprises compressing a helical spring of the filter device.

22. The method of claim 18, wherein said enlarging comprises twisting a helical spring of the filter device.

23. The method of claim 18, further comprising aspirating material concentrically around the at least one needle.

24. The method of claim 18, further comprising alternately flushing a region including the at least one needle and aspirating the region including the at least one needle.

25. The method of claim 18, further comprising:
aspirating a material proximate the at least one needle; and
sensing a condition selected from a pressure of the aspirated material and a flow rate of the aspirated material.

26. The method of claim 18, further comprising aspirating a material through at least one aspiration port through an exterior surface of the filter device.

27. An apparatus comprising:
a cannula to be percutaneously advanced through a blood vessel, the cannula having a proximal portion and a distal portion;
at least one needle coupled to the distal portion of the cannula;
at least one lumen through the cannula from the proximal portion to the at least one needle to provide at least one therapeutic agent to the at least one needle;
at least one aspiration port proximate the at least one needle; and
at least one lumen through the cannula from the proximal portion to the at least one aspiration port to convey suction provided at the proximal portion of the cannula to the at least one aspiration port.

28. The apparatus of claim 27, wherein the at least one aspiration port comprises at least one aspiration port on a distal end face of the cannula where the at least one needle is coupled.

29. The apparatus of claim 27, wherein the at least one aspiration port comprises a plurality of aspiration ports distributed around the at least one needle.

30. The apparatus of claim 29, wherein the plurality of aspiration ports are distributed concentrically around the at least one needle at a distal end face of the cannula where the at least one needle is coupled.

31. The apparatus of claim 29, wherein the at least one aspiration port comprises a plurality of aspiration ports distributed along a distal segment of the cannula.

32. The apparatus of claim 27, wherein the at least one aspiration port comprises an aspiration/flush port, and wherein the aspiration/flush port is alternatively coupled with a source of pressure to flow a fluid outwardly through the aspiration/flush port and with a suction to draw a material inwardly through the aspiration/flush port.

33. The apparatus of claim 27, further comprising at least one sensor to sense at least one of pressure of an aspirated fluid and flow rate of the aspirated fluid.
